(19) United States
(12) Patent Application Publication
(10) Pub. No.: US 2025/0049591 A1
(43) Pub. Date: Feb. 13, 2025

(54) METHOD AND SYSTEM FOR DESIGNING A STENT

(71) Applicant: VITAA MEDICAL SOLUTIONS INC., Montréal (CA)

(72) Inventors: Harold WODLINGER, Toronto (CA); D. Randy MOORE, Chestermere (CA)

(21) Appl. No.: 18/719,698

(22) PCT Filed: Dec. 12, 2022
(86) PCT No.: PCT/IB2022/062063

§ 371 Date: Jun. 13, 2024

Related U.S. Application Data
(60) Provisional application No. 63/265,320, filed on Dec. 13, 2021.

(51) Int. Cl. A61F 2/82; G16H 30/20; G16H 30/40

(52) U.S. Cl. CPC A61F 2/82; G16H 30/20; G16H 30/40; A61F 2240/002

(57) ABSTRACT

There is described a computer-implemented method for designing a stent, comprising: providing for display a first image of a 3D model of at least a portion of a body part comprising an aneurysm; receiving a position for each one of a plurality of reference points chosen along the body part; inserting in the first image a visual marker at the position of each reference point, thereby obtaining a second image; providing the second image for display; for each reference point, calculating a respective diameter; calculating at least one length using the positions of the reference points; and outputting the respective diameter and at least one length.

### FIELD

The present technology pertains to the field of medical imaging, surgical planning, surgical execution and vasculature. More specifically, the present technology relates to a method of and a system for designing a stent.

### BACKGROUND

Measurement of the geometry of vascular tissues is required for pre-operative patient monitoring, and for planning surgical intervention, both open and percutaneous repair. Surgical planning is required to ensure that stents and grafts are properly sized and properly constructed for the individual patient anatomy. Mistakes in stent sizing and construction can result in delayed, inappropriate, or inaccurate procedures, or lead to serious complications including death.

Geometry measurement is generally based on 3D medical imaging, including CT, MRI, and 3D Ultrasound modalities. Typically, the vessel(s) of interest are segmented, either manually or automatically, and presented to the physician on a computer screen. The physician makes geometric measurements including diameters, angles, and lengths, often using electronic calipers. However, vascular tissues such as arteries are complex three-dimensional structures that curve and twist in multiple planes and are difficult to measure accurately on two-dimensional computer screens.

Physicians often employ 3D medical imaging programs to measure the geometry of the vessel(s) using a number of advanced imaging techniques designed to allow more accurate measurements. However, these advanced imaging techniques are extremely complex and time consuming to use, and are prone to human error.

Therefore there is a need for an improved method and system for designing a stent.

### SUMMARY

It is an object of the present technology to ameliorate at least some of the inconveniences present in the prior art. One or more embodiments of the present technology may provide and/or broaden the scope of approaches to and/or methods of achieving the aims and objects of the present technology.

Developers of the present technology have appreciated that present techniques for designing a stent may be inaccurate since they rely on a medical practitioner for measuring parameters of a subject anatomy. However, vascular tissues such as arteries are complex three-dimensional structures that curve and twist in multiple planes and are difficult to measure accurately on a 2D computer screen.

The present method and system allow a much faster stent planning with substantially no loss in accuracy. They further allow a stent planning system that does not rely on curved or multi planar reconstruction, does not distort the 3D anatomy familiar to the medical practitioners, does not rely on electronic calipers, and is highly automated thereby minimizing user effort. This results in fewer errors in the design of a stent and better patient outcomes.

According to a first broad aspect, there is provided a computer-implemented method for designing a stent, the computer-implemented method comprising: providing for display a first image of a 3D model of a body part of a subject, the body part comprising at least an infrarenal segment of a thoracic aorta, an aneurysm on the infrarenal segment of the thoracic aorta, an aortic bifurcation and at least a portion of a left common iliac artery and a right common iliac artery; receiving a position for each one of a plurality of reference points, the reference points comprising at least an inflection point of the suprarenal segment, a bottom of a neck of the aneurysm, a bottom of the aneurysm, a first point and a second point on the left common iliac artery and a first and a second point on the right common iliac artery; inserting in the first image a visual marker at the position for each reference point, thereby obtaining a second image; providing the second image for display; for each reference point, calculating a respective diameter; calculating a first length of the neck using the position of the inflection point of the suprarenal segment and the position of the bottom of a neck of the aneurysm, a second length of a left landing area using the position of the first point and second points on the left common iliac artery, a third length of a right landing area using the position of the first point and second points on the right common iliac artery, a fourth length of a left leg of the stent and a fifth length of a right leg of the stent using the position of some of the reference points; calculating a suprarenal neck angle using the position of the inflection point of the suprarenal segment and the position of the bottom of the neck of the aneurysm, and an infrarenal neck angle using the position of the bottom of the neck of the aneurysm and the position of the bottom of the aneurysm; and outputting the respective diameter for each reference point, the first, second, third, fourth and fifth lengths, the suprarenal neck angle and the infrarenal neck angle.

In one embodiment, the visual marker is orthogonal to a center line.

In one embodiment, the fourth length is determined using the position of one of the inflection point and the bottom of the neck and the position of one of the first point and the second point on the left common iliac artery, and the fifth length is determined using the position of one of the inflection point and the bottom of the neck and the position of one of the first point and the second point on the right common iliac artery.

In one embodiment, the method further comprises: generating an image of a stent using the respective diameter for each reference point, the first, second, third, fourth and fifth lengths, the suprarenal neck angle, the infrarenal neck angle and a given predefined length for a body of the stent; combining the image of the stent on the image with the first image, thereby obtaining a further image; and providing the further image for display.

In one embodiment, the method further comprises receiving a position of a bifurcation point between the left and right common iliac arteries.

In one embodiment, the method further comprises calculating and outputting a diameter of the infrarenal segment at the bifurcation point.

In one embodiment, the method further comprises determining a sixth length using the position of one of the inflection point and the bottom of the neck and the position of the bifurcation point, said calculating the fourth length being performed using the position of the bifurcation point and the position of one of the first point and the second point on the left common iliac artery, and said calculating the fifth length being performed using the position of the bifurcation point and the position of one of the first point and the second point on the right common iliac artery.

In one embodiment, the method further comprises: generating an image of a stent using the respective diameter for each reference point, the first, second, third, fourth, fifth and sixth lengths, the suprarenal neck angle and the infrarenal neck angle; combining the image of the stent on the image with the first image, thereby obtaining a further image; and providing the further image for display.

In one embodiment, the step of outputting comprises providing the respective diameter for each reference point, the first, second, third, fourth and fifth lengths, the suprarenal neck angle and the infrarenal neck angle for display.

In one embodiment, the method further comprises: receiving a new position for the visual marker; moving the visual marker at the new position within the second image, thereby obtaining an updated image; providing the updated image for display; and calculating a further diameter at the new position.

In one embodiment, the visual marker at the new position is orthogonal to the center line.

In one embodiment, the method further comprises providing the further diameter for display.

In one embodiment, the method further comprises: generating a reference image of the body part, the reference image comprising at least a portion of a stylized infrarenal segment of the thoracic aorta, a stylized aneurysm, a stylized aortic bifurcation and at least a portion of a first stylized common iliac artery and a second stylized common iliac artery; inserting a visual indicator for each one of the plurality of the reference points within the reference image; and providing the reference image for display.

In one embodiment, the reference image further comprises a plurality of interactive boxes each associated with a respective reference point.

In one embodiment, the step of inserting in the first image the visual marker is performed only after receiving an indication that a given one of the interactive boxes has been selected.

In one embodiment, the 3D model comprises one of a RAW map, a strain map, an anatomy map, an ILT map and a growth map.

In one embodiment, the respective diameter comprises one of a maximal diameter, a minimal diameter and a given diameter of an equivalent circle.

In one embodiment, the method further comprises, for each one of the first and second common iliac arteries; receiving a respective iliac point; and calculating a respective curvature angle at the respective iliac point.

In one embodiment, the method further comprises providing the respective curvature angle for display.

In one embodiment, the method further comprises receiving an identification of the 3D model and retrieving the 3D model from a database.

In one embodiment, the body part further comprises at least a portion of a suprarenal segment of the thoracic aorta.

In one embodiment, the method further comprises: calculating a maximal diameter of the aneurysm; and providing the maximal diameter of the aneurysm for display.

In one embodiment, the step of generating the image of the stent comprises: accessing a database comprising predefined stent pieces; selecting adequate stent pieces amongst the predefined stent pieces using the respective diameter for each reference point, the first, second, third, fourth and fifth lengths, the suprarenal neck angle, the infrarenal neck angle and the given predefined length for the body of the stent, the adequate stent pieces forming together the stent; and generating the image of the stent using images of the adequate stent pieces.

In one embodiment, the step of generating the image of the stent comprises: accessing a database comprising predefined stent pieces; selecting adequate stent pieces amongst the predefined stent pieces using the respective diameter for each reference point, the first, second, third, fourth, fifth and sixth lengths, the suprarenal neck angle and the infrarenal neck angle, the adequate stent pieces forming together the stent; and generating the image of the stent using images of the adequate stent pieces.

According to another broad aspect, there is provided a computer-implemented method for designing a stent, comprising: providing for display a first image of a 3D model of at least a portion of a body part comprising an aneurysm; receiving a position for each one of a plurality of reference points chosen along the body part; inserting in the first image a visual marker at the position of each reference point, thereby obtaining a second image; providing the second image for display; for each reference point, calculating a respective diameter; calculating at least one length using the positions of the reference points; and outputting the respective diameter and at least one length.

According to a further broad aspect, there is provided a system for designing a stent, the system comprising: a processor; and a non-transitory computer readable storage medium comprising instructions stored thereon: the processor, upon execution of the instructions, being configured for: providing for display a first image of a 3D model of a body part of a subject, the body part comprising at least an infrarenal segment of a thoracic aorta, an aneurysm on the infrarenal segment of the thoracic aorta, an aortic bifurcation and at least a portion of a left common iliac artery and a right common iliac artery; receiving a position for each one of a plurality of reference points, the reference points comprising at least an inflection point of the suprarenal segment, a bottom of a neck of the aneurysm, a bottom of the aneurysm, a first point and a second point on the left common iliac artery and a first and a second point on the right common iliac artery; inserting in the first image a visual marker at the position for each reference point, thereby obtaining a second image; providing the second image for display; for each reference point, calculating a respective diameter; calculating a first length of the neck using the position of the inflection point of the suprarenal segment and the position of the bottom of a neck of the aneurysm, a second length of a left landing area using the position of the first point and second points on the left common iliac artery, a third length of a right landing area using the position of the first point and second points on the right common iliac artery, a fourth length of a left leg of the stent and a fifth length of a right leg of the stent using the position of some of the reference points; calculating a suprarenal neck angle using the position of the inflection point of the suprarenal segment and the position of the bottom of the neck of the aneurysm, and an infrarenal neck angle using the position of the bottom of the neck of the aneurysm and the position of the bottom of the aneurysm; and outputting the respective diameter for each reference point, the first, second, third, fourth and fifth lengths, the suprarenal neck angle and the infrarenal neck angle.

In one embodiment, the visual marker is orthogonal to a center line.

In one embodiment, the fourth length is determined using the position of one of the inflection point and the bottom of the neck and the position of one of the first point and the second point on the left common iliac artery, and the fifth length is determined using the position of one of the inflection point and the bottom of the neck and the position of one of the first point and the second point on the right common iliac artery.

In one embodiment, the processor is further configured for generating an image of a stent using the respective diameter for each reference point, the first, second, third, fourth and fifth lengths, the suprarenal neck angle, the infrarenal neck angle and a given predefined length for a body of the stent; combining the image of the stent on the image with the first image, thereby obtaining a further image; and providing the further image for display.

In one embodiment, the processor is further configured for receiving a position of a bifurcation point between the left and right common iliac arteries.

In one embodiment, the processor is further configured for calculating and outputting a diameter of the infrarenal segment at the bifurcation point.

In one embodiment, the processor is further configured for determining a sixth length using the position of one of the inflection point and the bottom of the neck and the position of the bifurcation point, said calculating the fourth length being performed using the position of the bifurcation point and the position of one of the first point and the second point on the left common iliac artery, and said calculating the fifth length being performed using the position of the bifurcation point and the position of one of the first point and the second point on the right common iliac artery.

In one embodiment, the processor is further configured for: generating an image of a stent using the respective diameter for each reference point, the first, second, third, fourth, fifth and sixth lengths, the suprarenal neck angle and the infrarenal neck angle; combining the image of the stent on the image with the first image, thereby obtaining a further image; and providing the further image for display.

In one embodiment, said outputting comprises providing the respective diameter for each reference point, the first, second, third, fourth and fifth lengths, the suprarenal neck angle and the infrarenal neck angle for display.

In one embodiment, the processor is further configured for: receiving a new position for the visual marker; moving the visual marker at the new position within the second image, thereby obtaining an updated image; providing the updated image for display; and calculating a further diameter at the new position.

In one embodiment, the visual marker at the new position is orthogonal to the center line.

In one embodiment, the processor is further configured for providing the further diameter for display.

In one embodiment, the processor is further configured for: generating a reference image of the body part, the reference image comprising at least a portion of a stylized infrarenal segment of the thoracic aorta, a stylized aneurysm, a stylized aortic bifurcation and at least a portion of a first stylized common iliac artery and a second stylized common iliac artery; inserting a visual indicator for each one of the plurality of the reference points within the reference image; and providing the reference image for display.

In one embodiment, the reference image further comprises a plurality of interactive boxes each associated with a respective reference point.

In one embodiment, said inserting in the first image the visual marker is performed only after receiving an indication that a given one of the interactive boxes has been selected.

In one embodiment, the 3D model comprises one of a RAW map, a strain map, an anatomy map, an ILT map and a growth map.

In one embodiment, the respective diameter comprises one of a maximal diameter, a minimal diameter and a given diameter of an equivalent circle.

In one embodiment, the processor is further configured for, for each one of the first and second common iliac arteries; receiving a respective iliac point; and calculating a respective curvature angle at the respective iliac point.

In one embodiment, the processor is further configured for providing the respective curvature angle for display.

In one embodiment, the processor is further configured for receiving an identification of the 3D model and retrieving the 3D model from a database.

In one embodiment, the body part further comprises at least a portion of a suprarenal segment of the thoracic aorta.

In one embodiment, the processor is further configured for: calculating a maximal diameter of the aneurysm; and providing the maximal diameter of the aneurysm for display.

In one embodiment, said generating the image of the stent comprises: accessing a database comprising predefined stent pieces; selecting adequate stent pieces amongst the predefined stent pieces using the respective diameter for each reference point, the first, second, third, fourth and fifth lengths, the suprarenal neck angle, the infrarenal neck angle and the given predefined length for the body of the stent, the adequate stent pieces forming together the stent; and generating the image of the stent using images of the adequate stent pieces.

In one embodiment, said generating the image of the stent comprises: accessing a database comprising predefined stent pieces; selecting adequate stent pieces amongst the predefined stent pieces using the respective diameter for each reference point, the first, second, third, fourth, fifth and sixth lengths, the suprarenal neck angle and the infrarenal neck angle, the adequate stent pieces forming together the stent; and generating the image of the stent using images of the adequate stent pieces.

According to still another broad aspect, there is provided a system for designing a stent, the system comprising: a processor; and a non-transitory computer readable storage medium comprising instructions stored thereon: the processor, upon execution of the instructions, being configured for: providing for display a first image of a 3D model of at least a portion of a body part comprising an aneurysm; receiving a position for each one of a plurality of reference points chosen along the body part; inserting in the first image a visual marker at the position of each reference point, thereby obtaining a second image; providing the second image for display; for each reference point, calculating a respective diameter; calculating at least one length using the positions of the reference points; and outputting the respective diameter and at least one length.

In the following, the expression “center line” refers to the center line of the anatomy, i.e., the center line of the wall of the aorta or artery. In an embodiment in which a thrombus is present, the center line refers to the center line of the lumen.

### Terms and Definitions

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression “a server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, unless expressly provided otherwise, a computer system may refer, but is not limited to, an “electronic device”, a “computing device”, an “operation system”, a “system”, a “computer-based system”, a “computer system”, a “network system”, a “network device”, a “controller unit”, a “monitoring device”, a “control device”, a “server”, and/or any combination thereof appropriate to the relevant task at hand.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, cloud storage, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e. its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the servers, nor is their use (by itself) intended to imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 depicts a schematic diagram of an electronic device in accordance with one or more non-limiting embodiments of the present technology;

FIG. 2 depicts a schematic diagram of a system in accordance with one or more non-limiting embodiments of the present technology;

FIG. 3 is a flow chart illustrating a computer-implemented method for designing a stent, in accordance with one or more non-limiting embodiments of the present technology;

FIG. 4 is an image of an anatomy part comprising an aorta provided with an aneurysm and two common iliac arteries, in accordance with one or more non-limiting embodiments of the present technology;

FIG. 5 corresponds to the image of the aorta of FIG. 4 in which a first reference marker has been added, in accordance with one or more non-limiting embodiments of the present technology;

FIG. 6 corresponds to the image of the aorta of FIG. 4 in which nine reference markers have been added, in accordance with one or more non-limiting embodiments of the present technology;

FIG. 7 illustrates the image of the aorta of FIG. 4 in which the suprarenal and infrarenal angles of the neck of the aneurysm are shown, in accordance with one or more non-limiting embodiments of the present technology;

FIG. 8 illustrates a first rotation of the aorta of FIG. 4, in accordance with one or more non-limiting embodiments of the present technology;

FIG. 9 illustrates a first rotation of the aorta of FIG. 4, in accordance with one or more non-limiting embodiments of the present technology;

FIG. 10 illustrates a cross-sectional view of the aorta of FIG. 4, in accordance with one or more non-limiting embodiments of the present technology;

FIG. 11 illustrates the curvature of the two common iliac arteries of FIG. 4, in accordance with one or more non-limiting embodiments of the present technology;

FIG. 12 is an image comprising the anatomy part of FIG. 4 and a stylized representation of the anatomy part, in accordance with one or more non-limiting embodiments of the present technology;

FIG. 13 corresponds to the image of FIG. 12 in which a reference marker has been added on the image of the anatomy part, in accordance with one or more non-limiting embodiments of the present technology;

FIG. 14 corresponds to the image of FIG. 12 in which nine reference markers have been added on the image of the anatomy part, in accordance with one or more non-limiting embodiments of the present technology;

FIG. 15 illustrates the anatomy part of FIG. 4 in which an image of a designed stent has been incorporated, in accordance with one or more non-limiting embodiments of the present technology; and

FIG. 16 illustrates a stent for a thoracic aortic aneurysm inserted into a thoracic aorta, in accordance with one or more non-limiting embodiments of the present technology.

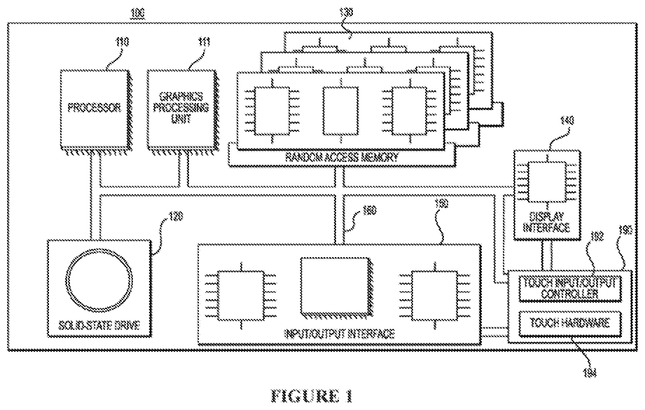

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some non-limiting embodiments of the present technology, the processor may be a general-purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, we will now consider some non-limiting examples to illustrate various implementations of aspects of the present technology.

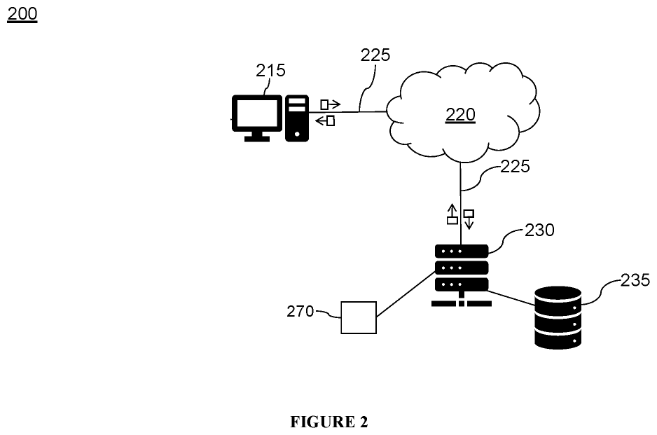

With reference to FIG. 2, there is illustrated a schematic diagram of an electronic device 100 suitable for use with some non-limiting embodiments of the present technology.

### Electronic Device

The electronic device 100 comprises various hardware components including one or more single or multi-core processors collectively represented by processor 110, a graphics processing unit (GPU) 111, a solid-state drive 120, a random-access memory 130, a display interface 140, and an input/output interface 150.

Communication between the various components of the electronic device 100 may be enabled by one or more internal and/or external buses 160 (e.g. a PCI bus, universal serial bus, IEEE 1394 “Firewire” bus, SCSI bus, Serial-ATA bus, etc.), to which the various hardware components are electronically coupled.

The input/output interface 150 may be coupled to a touchscreen 190 and/or to the one or more internal and/or external buses 160. The touchscreen 190 may be part of the display. In some embodiments, the touchscreen 190 is the display. The touchscreen 190 may equally be referred to as a screen 190. In the embodiments illustrated in FIG. 2, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the electronic device 100 in addition or in replacement of the touchscreen 190.

According to implementations of the present technology, the solid-state drive 120 stores program instructions suitable for being loaded into the random-access memory 130 and executed by the processor 110 and/or the GPU 111 for training and using machine learning models for pre-interventional planning and post-interventional monitoring of endovascular aortic repair (EVAR). For example, the program instructions may be part of a library or an application.

The electronic device 100 may be implemented in the form of a server, a desktop computer, a laptop computer, a tablet, a smartphone, a personal digital assistant or any device that may be configured to implement the present technology, as it may be understood by a person skilled in the art.

### System

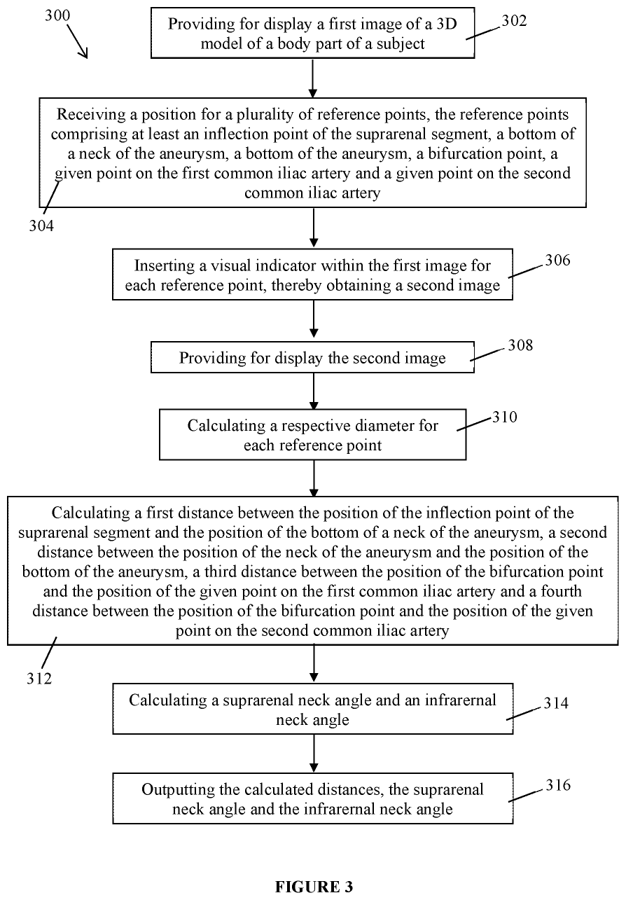

Referring to FIG. 3, there is shown a schematic diagram of a communication system 200, which will be referred to as the system 200, the system 200 being suitable for implementing non-limiting embodiments of the present technology. It is to be expressly understood that the system 200 as illustrated is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition it is to be understood that the system 200 may provide in certain instances simple implementations of the present technology, and that where such is the case they have been presented in this manner as an aid to understanding. As persons skilled in the art would understand, various implementations of the present technology may be of a greater complexity.

The system 200 comprises inter alia a workstation computer 215, a server 230 and a database 235 coupled over a communications network 220 via respective communication links 225 (not separately numbered).

In one or more embodiments, at least a portion of the system 200 implements the Picture Archiving and Communication System (PACS) technology.

### Workstation Computer

The workstation computer 215 is configured to allow the user designing a stent.

The implementation of the workstation computer 215 is known in the art. The workstation computer 215 may be implemented as the electronic device 100 or comprise components thereof, such as the processor 110, the graphics processing unit (GPU) 111, the solid-state drive 120, the random-access memory 130, the display interface 140, and the input/output interface 150.

In one or more embodiments, the workstation computer 215 is configured according to the Digital Imaging and Communications in Medicine (DICOM) standard for communication and management of medical imaging information and related data.

In one or more embodiments, the workstation computer 215 may store the images in a local database (not illustrated).

The workstation computer 215 is connected to a server 230 over the communications network 220 via a respective communication link 225. In one or more embodiments, the workstation computer 215 may transmit the images and/or multiphase stack to the server 230 and/or the database 235 for storage and/or processing thereof.

### Server

In one or more embodiments of the present technology, the server 230 is configured to inter alia;
provide for display a first image of a 3D model of a body part of a subject, the body part comprising at least a portion of an infrarenal segment of a thoracic aorta, an aneurysm on the infrarenal segment of the thoracic aorta, an aortic bifurcation and at least a portion of a left common iliac artery and a right common iliac artery;
receive a position for each one of a plurality of reference points, the reference points comprising at least an inflection point of a suprarenal segment, a bottom of a neck of the aneurysm, a bottom of the aneurysm, a first point and a second point on the left common iliac artery and a first and a second point on the right common iliac artery;
insert in the first image a visual marker at the position for each reference point, thereby obtaining a second image;
provide the second image for display;
for each reference point, determine a respective diameter;
determine a first length of the neck using the position of the inflection point of the suprarenal segment and the position of the bottom of a neck of the aneurysm, a second length of a left landing area using the position of the first point and second points on the left common iliac artery, a third length of a right landing area using the position of the first point and second points on the right common iliac artery, a fourth length of a left leg of the stent and a fifth length of a right leg of the stent using the position of some of the reference points;
determine a suprarenal neck angle using the position of the inflection point of the suprarenal segment (i.e. where the suprarenal segment of the aorta appears to bend) and the position of the bottom of the neck of the aneurysm, and an infrarenal neck angle using the position of the bottom of the neck of the aneurysm and the position of the bottom of the aneurysm; and
output the respective diameter for each reference point, the first, second, third, fourth and fifth lengths, the suprarenal neck angle and the infrarenal neck angle.

How the server 230 is configured to do so will be explained in more detail herein below.

The server 230 can be implemented as a conventional computer server and may comprise some or all of the components of the electronic device 100 illustrated in FIG. 2. In an example of one or more embodiments of the present technology, the server 230 can be implemented as a Dell™ PowerEdge™ Server running the Microsoft™ Windows Server™ operating system. Needless to say, the server 230 can be implemented in any other suitable hardware and/or software and/or firmware or a combination thereof. In the illustrated non-limiting embodiment of present technology, the server 230 is a single server. In alternative non-limiting embodiments of the present technology, the functionality of the server 230 may be distributed and may be implemented via multiple servers (not illustrated).

The implementation of the server 230 is well known to the person skilled in the art of the present technology. However, briefly speaking, the server 230 comprises a communication interface (not illustrated) structured and configured to communicate with various entities (such as the workstation computer 215, for example and other devices potentially coupled to the network 220) via the communications network 220. The server 230 further comprises at least one computer processor (e.g., a processor 110 or GPU 111 of the electronic device 100) operationally connected with the communication interface and structured and configured to execute various processes to be described herein.

In one or more embodiments, the server 230 may be implemented as the electronic device 100 or comprise components thereof, such as the processor 110, the graphics processing unit (GPU) 111, the solid-state drive 120, the random-access memory 130, the display interface 140, and the input/output interface 150.

It will be appreciated that the server 230 may provide the output of one or more processing steps to another electronic device for display, confirmation and/or troubleshooting. As a non-limiting example, the server 230 may transmit the maps (e.g., strain map, RAW map, ILT map, etc.) and results of assessments (pre-EVAR and post-EVAR) for display on a client device configured similar to the electronic device 100 such as a smart phone, tablet, and the like.

### Database

The database 235 is configured to inter alia; (i) store DICOM stacks; (ii) store anatomy 3D models; (iii) images; and/or (iv) store predefined parameter or characteristic values.

In one or more embodiments, the database 235 may store ML file formats, such as .tfrecords, .csv, .npy, and .petastorm as well as the file formats used to store models, such as .pb and .pkl. In one or more embodiments, the database 235 may also store well-known file formats such as, but not limited to image file formats (e.g., .png, .jpeg), video file formats (e.g., .mp4, .mkv, etc), archive file formats (e.g., .zip, .gz, .tar, .bzip2), document file formats (e.g., .docx, .pdf, .txt) or web file formats (e.g., .html).

It will be appreciated that the database 235 may store other types of data such as validation datasets (not illustrated), test datasets (not illustrated) and the like.

### Communication Network

In some embodiments of the present technology, the communications network 220 is the Internet. In alternative non-limiting embodiments, the communication network 220 can be implemented as any suitable local area network (LAN), wide area network (WAN), a private communication network or the like. It should be expressly understood that implementations for the communication network 220 are for illustration purposes only. How a communication link 225 (not separately numbered) between the workstation computer 215 and/or the server 230 and/or another electronic device (not illustrated) and the communications network 220 is implemented will depend inter alia on how each of the medical imaging apparatus 210, the workstation computer 215, and the server 230 is implemented.

The communication network 220 may be used in order to transmit data packets amongst the workstation computer 215, the server 230 and the database 235. For example, the communication network 220 may be used to transmit requests between the workstation computer 215 and the server 230.

### Method Description

FIG. 3 depicts a flowchart of a method 300 for designing a stent in accordance with one or more non-limiting embodiments of the present technology. The stent is to be inserted into an abdominal aorta of a subject for treatment of an aneurysm. The method 300 is used by a medical practitioner such as a physician, a surgeon, or the like to determine relevant characteristics of an anatomy of a subject that are to be used in the design of the stent. For example, the method 300 allows a medical practitioner to precisely determine diameters, angles, distances, etc. of the anatomy.

In one or more embodiments, the server 230 comprises a processing device such as the processor 110 and/or the GPU 111 operatively connected to a non-transitory computer readable storage medium such as the solid-state drive 120 and/or the random-access memory 130 storing computer-readable instructions. The processing device, upon executing the computer-readable instructions, is configured to or operable to execute the method 300.

The method 300 begins at processing step 302. A first image 400 of a 3D model is provided for display to the user. The 3D model represents a part of the real anatomy of a subject. The body or anatomy part comprises at least a portion of the suprarenal segment 402 of a thoracic aorta of the subject, an aneurysm 404 present in the aorta, the aortic bifurcation 406 and at least a portion of the two common iliac arteries 408 and 410. As illustrated in FIG. 4. It should be understood that the portion of the suprarenal segment 402 may be omitted in the image 400 and/or the whole common iliac arteries 408 and 410 may be included in the image 400.

It should be understood that the image 400 is an image of the real anatomy of the subject. In one embodiment, the image 400 is a real size image of the anatomy.

In one embodiment, the image 400 is a 2D image of the 3D model. In another embodiment, the image 400 is a 3D image of the 3D model.

In one embodiment, the user may rotate the image 400 in order to see a different view of the represented anatomy.

Once the image 400 is displayed on a user display, the user selects the position for a plurality of reference points on the displayed image. The position of each selected point is then received at processing step 304.

It should be understood that any adequate method for selecting the position for a reference point can be used. For example, the user may use a mouse and right click on the displayed image 400 at the desired position for the reference point. In another example in which the image 400 is displayed on a touch screen, the user may touch the screen at the desired position for the reference point.

In one embodiment, the user already knowns which reference points has to be selected on the image. In the same or another embodiment, instructions defining the reference points to be selected may be provided to the user.

In one embodiment, the reference points to be selected by the user comprise at least the following reference point; the inflection point of the suprarenal segment, the bottom of the neck of the aneurysm, the bottom of the aneurysm, a first point and a second point on the left common iliac artery and a first point and a second point on the right common iliac artery. The inflection point of the suprarenal segment and the bottom of the neck of the aneurysm are chosen to define the landing area for the neck of the stent while the first and second points selected on the left common iliac artery are chosen to define the landing area for the left leg of the stent and the first and second points selected on the right common iliac artery are chosen to define the landing area for the right leg of the stent.

In this embodiment and as described below, a reference length for the body of the stent is stored in memory and used for designing the stent.

In one embodiment, the reference points further comprise the bifurcation point between the left and right common iliac arteries. In this embodiment, the bottom of the neck of the aneurysm and the bifurcation point are used to define the length of the body of the stent, e.g., the length of the body of the stent is chosen based on the positions of the bottom of the neck of the aneurysm and the bifurcation point. In one embodiment, the length of the body of the stent is chosen to be less than the distance between the bottom of the neck of the aneurysm and the bifurcation point.

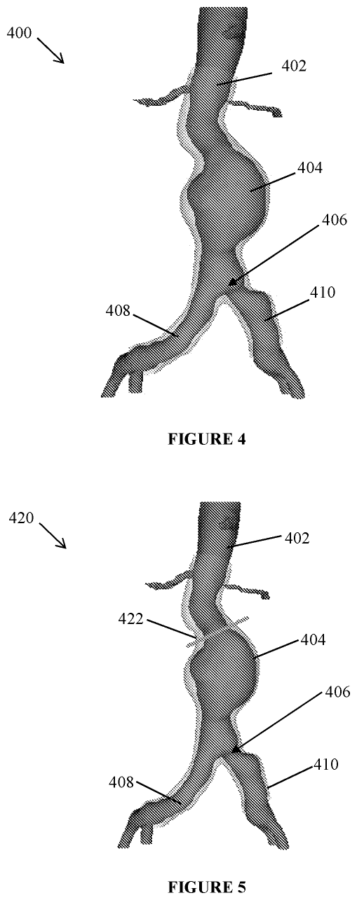

At processing step 304, for each reference point identified by the user, a respective visual marker is generated and inserted into the first image at the position received from the user. For example, FIG. 5 illustrates a second image 420 of the anatomy which corresponds to the first image 400 in which a visual marker 422 has been inserted at the position for the bottom of the neck of the aneurysm selected by the user. The bottom of the neck defines the bottom of the landing area for the stent.

In the illustrated embodiment, the visual marker 422 is a line orthogonal to the center line of the aorta at the selected position. In an embodiment in which the image 400 is a 3D image, the visual marker may be a plate or portion of a plane orthogonal to the center line of the aorta at the selected position. It should be understood that any adequate visual marker allowing to visualize a selected position in an image may be used.

Once it has been generated, the second image 420 is provided for display. It should be understood that the second image 422 is displayed to the user in replacement of the first image 400.

It should be understood that a visual marker such as visual marker 422 is inserted into the image 400 for each reference point at its respective received position selected by the user.

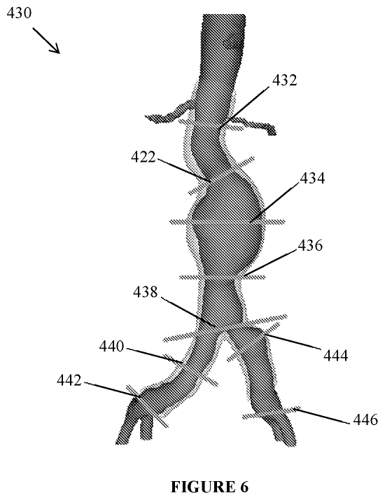

FIG. 6 illustrates a third image 430 of the anatomy which corresponds to the first image 400 in which nine visual markers 422 and 432-446 have been added each at its respective position selected by the user. In the illustrated example, the visual markers 422 and 432-446 each correspond to a line orthogonal to the center line of the anatomy at their respective position.

The visual marker 432 is positioned at the position selected by the user for the inflection point of the suprarenal segment of the thoracic aorta. The inflection point of the suprarenal segment defines the top of the landing area within the suprarenal segment of the aorta.

As described above, the visual marker 422 indicates the position of the bottom of a neck of the aneurysm selected by the user.

The visual marker 436 indicates the position selected by the user for the bottom of the aneurysm.

The visual marker 440 indicates the position selected for the first reference point on the left common iliac artery and the visual marker 442 indicates the position selected for the second reference point on the left common iliac artery. In the illustrated embodiment, the visual marker 442 is positioned at the bifurcation point of the left common iliac artery 408, i.e., the point at which the left common iliac 408 splits into the left internal iliac artery and the left external iliac artery.

The visual marker 444 indicates the position selected for the first reference point on the right common iliac artery and the visual marker 446 indicates the position selected for the second reference point on the right common iliac artery. In the illustrated embodiment, the visual marker 446 is positioned at the bifurcation point of the right common iliac artery 410, i.e., the point at which the right common iliac 410 splits into the right internal iliac artery and the right external iliac artery.

Optionally, the user selects the position of the center of the aneurysm and the visual marker 434 is inserted at the selected position. The user may also select the position of the bifurcation point between the left and right common iliac arteries and the visual marker 438 is inserted into the image.

In one embodiment, a visual marker is inserted into the image 400 as soon as the position for a reference point is received from the user. In another embodiment, the visual markers are inserted into the image 400 once the position for all reference points has been received.

At processing step 310, for at least some of the reference points for which a respective position has been selected by the user, the diameter of the anatomy at the respective position orthogonal to the center line is determined using the 3D model of the anatomy. For example, the diameter of the infrarenal segment of the abdominal aorta is determined at the position received from the user for the inflection point of the suprarenal segment, i.e. the diameter of the cross-section of the infrarenal segment taken at the received position for the inflection point orthogonally to the center line of the infrarenal segment at that position is calculated.

In one embodiment, the diameter of the anatomy is determined at least for the following reference points; the inflection point of the suprarenal segment represented by the visual marker 432, the bottom of the neck of the aneurysm represented by the visual marker 422, the first and second points selected on the left common iliac artery represented by the visual markers 440 and 442, respectively, and the first and second points on the right common iliac artery represented by the visual markers 444 and 446.

In one embodiment, the diameter is also determined for the following further reference points; the bottom of the aneurysm represented by the visual marker 432, the center of the aneurysm represented by the visual marker 434 and/or the bifurcation point represented by the visual marker 438.

Since the anatomy is usually not symmetrical, the diameter of the anatomy may not be constant within a cross-section of the anatomy. In one embodiment, the diameter determined at processing step 310 and associated with a reference point corresponds to the maximal diameter of the cross-section at the position of the reference point. In another embodiment, the diameter determined at processing step 310 and associated with a reference point corresponds to the minimal diameter of the cross-section at the position of the reference point. In a further embodiment, the diameter determined at processing step 310 corresponds to a combination of the maximal and minimal diameters such as the mean of the maximal and minimal diameters. In still another embodiment, the diameter determined at processing step 310 corresponds to the diameter of an equivalent circle. In this case, processing step 310 comprises measuring the perimeter of the cross-section of the anatomy at the position of the reference point and determining the diameter of a circle of which the circumference is equal to the determined perimeter.

While in the present description, the diameter of the reference point is determined at processing step 310, the person skilled in the art will understand that processing step 310 may comprise determining the radius of the cross-section taken at the position of the reference points instead of the diameter since a diameter and a radius are equivalent.

At processing step 312, a plurality of distances or lengths between at least some reference points are calculated using the 3D model. In one embodiment, at least five lengths are calculated at processing step 312.

In one embodiment, the length or distance between two reference points is taken along the center line of the anatomy.

The first length corresponds to the length of the neck for the stent and corresponds to the distance between the position of the inflection point of the suprarenal segment identified by visual marker 432 in FIG. 6 and the position of the bottom of the neck of the aneurysm identified by visual marker 422.

The second length corresponds to the length of the landing area in the left common iliac artery and corresponds to the distance between the positions of first and second reference points on the left common iliac artery represented by the visual markers 440 and 442.

The third length corresponds to the length of the landing area in the right common iliac artery and corresponds to the distance between the positions of first and second reference points on the right common iliac artery represented by the visual markers 444 and 446.

The fourth length is associated with the left common iliac artery and corresponds to the distance between the position of the inflection point of the suprarenal segment identified by visual marker 432 or the position of the bottom of the neck of the aneurysm identified by visual marker 422 and the position of one of the two reference points on the left common iliac artery identified by visual markers 440 and 442.

The fifth length is associated with the right common iliac artery and corresponds to the distance between the position of the inflection point of the suprarenal segment identified by visual marker 432 or the position of the bottom of the neck of the aneurysm identified by visual marker 422 and the position of one of the two reference points on the right common iliac artery identified by visual markers 444 and 446.

In an embodiment in which the position of the bifurcation point identified by the visual marker 438 has been selected by the user, a sixth length may be determined. The sixth length corresponds to the distance between the position of the bifurcation point and the position of the inflection point of the suprarenal segment identified by visual marker 432 or the position of the bottom of the neck of the aneurysm identified by visual marker 422 is further determined. In this case, the fourth length is associated with the left common iliac artery may correspond to the distance between the position of the bifurcation point and the position of one of the two reference points on the left common iliac artery identified by visual markers 440 and 442. Similarly, the fifth length is associated with the right common iliac artery corresponds to the distance between the position of the bifurcation point and the position of one of the two reference points on the right common iliac artery identified by visual markers 444 and 446.

At processing step 314, the suprarenal neck angle α and the infrarenal neck angle β are determined using the 3D model. More precisely, the suprarenal neck angle α is determined using the position of the inflection point of the suprarenal segment and the position of the bottom of the neck of the aneurysm, and the infrarenal neck angle β is determined using the position of the inflection point of the suprarenal segment, the position of the bottom of the neck of the aneurysm and the position of the bottom of the aneurysm as described below.

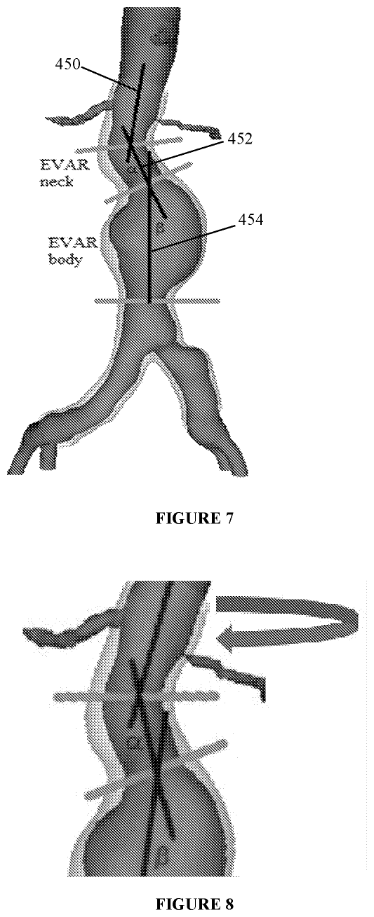

As illustrated in FIG. 7, the suprarenal neck angle α corresponds to the angle between the center line 450 of the suprarenal segment and the axis 452 that passes by the center of the cross-section taken at the position of the inflection point and the center of the cross-section taken at the position of the bottom of the neck of the aneurysm. The infrarenal neck angle β corresponds to the angle between the axis 452 and the axis 454 that passes by the center of the cross-section taken at the position of the bottom of the neck of the aneurysm and the center of the cross-section of taken at the position of the bottom of the aneurysm.

In an embodiment in which the value of the suprarenal neck angle α and that of the infrarenal neck angle β vary depending on the view of the 3D model, the most severe or greatest suprarenal neck angle α and the most severe or greatest infrarenal neck angle β are determined at processing step 314.

In one embodiment, the most severe suprarenal neck angle α and infrarenal neck angle β are determined as follows.

In order to determine the most severe suprarenal neck angle α, the 3D model is rotated about the visual marker 432 to obtain a view perpendicular to the middle of the suprarenal neck angle α, then the 3D model is rotated 360° about the axis 452 and the most severe angle α between axes 452 and 450 over the 360° is taken as the suprarenal neck angle α, as illustrated in FIG. 8.

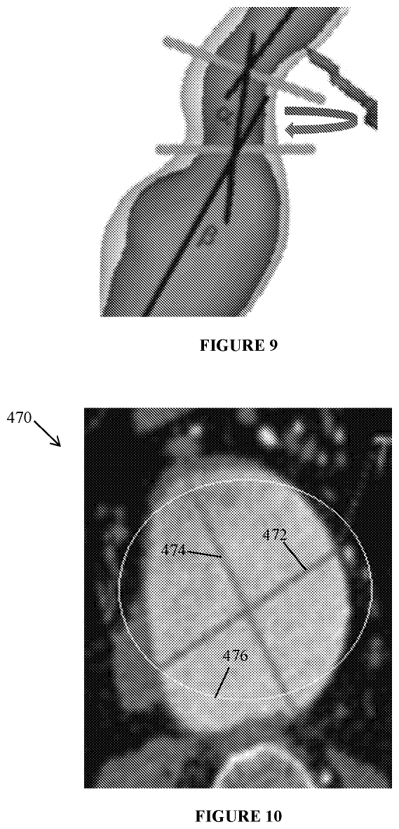

The most severe infrarenal neck angle β is determined in a similar manner. the 3D model is rotated about the visual marker 422 to obtain a view perpendicular to the middle of the infrarenal neck angle β, then the 3D model is rotated 360° about the axis 452 and the most severe angle β between axes 452 and 454 over the 360° is taken as the infrarenal neck angle β, as illustrated in FIG. 9.

At processing step 316, the determined parameters, i.e., the diameters determined at processing step 310, the distances determined at processing step 312 and the angles determined at processing step 314, are outputted.

In one embodiment, the determined parameters are stored in memory. In the same or another embodiment, the determined parameters are provided for display.

The method 300 then ends.

In one embodiment, the reference points comprise at least the inflection point of the suprarenal segment, the bottom of the neck of the aneurysm, the bottom of the aneurysm, the bifurcation point, a given point on the first common iliac artery and a given point on the second common iliac artery. However, it should be understood that the reference points may comprise more than the above-listed points.

It should be understood that the order in which the processing steps 310-316 are executed may vary. For example, each time the position of a reference point is received from the user, the diameter of the anatomy associated with the reference point may be determined and outputted before the reception of the position of another reference point. Alternatively, the diameters may be displayed to the user only when the position for all of the reference points has been received from the user. In another example, as soon as they can be determined, a distance between two reference points and/or the suprarenal neck angle α and/or the infrarenal neck angle β are determined and outputted. Alternatively, the determined distances and/or the determined angles can be displayed to the user only when all distances and angles have been determined. Similarly, a visual marker may be inserted into the image 400 and the updated image may be displayed to the user as soon as the position of its respective reference point is received. Alternatively, the visual markers may be displayed to the user only once the position for all reference points has been received.

In one embodiment, the visual markers 422 and 432-446 are each interactive, i.e., the user may select a visual marker and move it to another position to define a better position for the reference point associated thereto. In this case, the new position of the visual marker, which defines the position for the reference point associated with the reference marker, is transmitted to the server 230 and the method 300 further comprises the processing step of receiving the new position for the reference point. The method further comprises calculating the parameters, e.g., the diameter of the anatomy at the new position, using the new position and outputting the new value of the parameters.

In an embodiment in which a visual marker 422, 432-446 is interactive, the marker is generated and displayed to the user so as to be orthogonal to the center line. Since the visual marker 422, 432-446 is interactive, the user may move the visual marker 422, 432-446 from an initial position to a second position. In one embodiment, when at the final position, the visual marker 422, 432-446 is positioned within the displayed image so at to be orthogonal to the center line. In one embodiment, the visual marker 422, 432-446 is displayed to the user while the user moves the visual marker 422, 432-446 from the initial position to the final position, i.e., the visual marker 422, 432-446 is displayed to the user at each intermediary position between the initial and final positions. In one embodiment, for each intermediary position, the visual marker 422, 432-446 is positioned within the displayed image so as to be orthogonal to the center line, i.e. the visual marker 422, 432-446 is forced to remain orthogonal to the center line while the user is moving the visual marker 422, 432-446.

In one embodiment, the user may display a cross-sectional view of the anatomy at a position where the diameter has to be determined and the cross-sectional view may be taken orthogonal to the center line. For example, the user may select the visual marker 422 and display the cross-sectional view of the anatomy at this location. In this case, the method 300 comprises a processing step of generating the cross-sectional view of the 3D model at the location indicated by the user, e.g., at the location corresponding to the visual marker 422. FIG. 10 illustrates an exemplary cross-sectional view 470 taken at the position indicated by the user, in which visual markers 472, 474 and/or 476 are added before being displayed to the user. The visual marker 472 corresponds to the smallest diameter of the cross-section of the aorta at the position indicated by the user. The visual marker 474 corresponds to the longest diameter of the cross-section of the aorta at the position indicated by the user. The visual diameter 476 is a circle centered on the center of the cross-section of the aorta and of which the circumference corresponds to the perimeter of the cross-section. In one embodiment, only one of the visual markers 472-476 may be generated. In another embodiment, only two of the visual markers 472-476 may be generated.

The visual markers 472-476 are interactive, i.e., the user may modify them. For example, the user may enlarge the circle 476 and the new diameter of the circle 476 is determined and outputted as the new diameter associated with the position selected by the user.

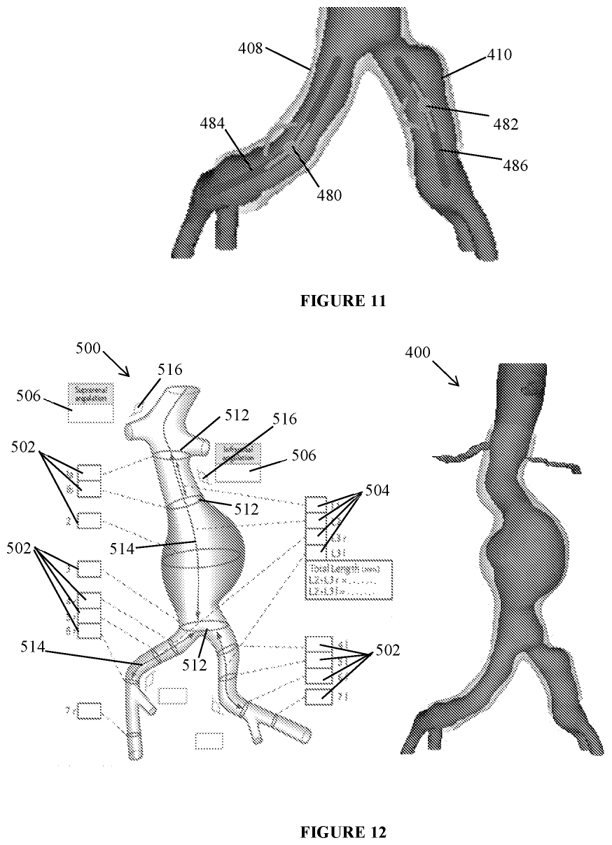

In one embodiment, the method 300 further comprises determining the curvature of the first and/or second common iliac arteries 408 and 410. In this case, the method 300 further comprises a processing step of receiving from the user the position of a reference point 480, 482 on the common iliac artery 408, 410 and a processing step of determining the curvature of the center line 484, 486 at point 480, 482, as illustrated in FIG. 11. The method may further comprise the step of outputting the determined curvature.

It should be understood that other curvatures of the anatomy may be determined. For example, the outer and/or inner curvature of the aortic neck and/or the aortic sac may be determined and outputted.

In one embodiment, the method further comprises adding a visual marker at the position selected by the user and/or the center line in the image 400 to help the user visualize the reference point that he selected and/or the center line.

In one embodiment, in addition to the generation and display of the image 400 of the 3D model, the method 300 further comprises the processing step of providing for display an image a reference image 500. As illustrated in FIG. 12, the reference image 500 comprises a stylized representation of the same body part as that illustrated in the image 400, i.e., a representation of at least a portion of a stylized suprarenal segment of the thoracic aorta, a stylized aneurysm, a stylized aortic bifurcation and at least a portion of a first stylized common iliac artery and a second stylized common iliac artery.

The image 500 further comprises a plurality of interactive diameter boxes 502, a plurality of interactive length or distance boxes 504 and a plurality of interactive angle boxes 506. The image 500 also comprises visual markers 512, 514 and 516 each being associated with a respective diameter box 502, distance box 504 or angle box 506.

Each diameter box 502 is associated with a respective reference point on the anatomy and a respective visual marker 512. The visual marker 512 associated with a given diameter box 502 is positioned in the image 500 at the position of the reference point associated with the given diameter box 502. Furthermore, each diameter box 502 and its respective visual marker 512 are linked by a dashed line links to help the user visualize the association between diameter boxes 502 and visual markers 512.

Each distance box 504 is associated with a respective distance or length between two respective reference points and a respective visual marker 514 in the form of an arrow between the two reference points. The visual marker 514, e.g., the arrow, associated with a given distance box 504 is positioned in the image 500 so as to extend between the two reference points associated with the distance box 504 along the center line of the anatomy. Furthermore, each distance box 504 and its respective visual marker 514 are linked by a dashed line links to help the user visualize the association between distance boxes 504 and visual markers 514.

Each angle box 506 is associated with a respective angle within the anatomy and a respective visual marker 516. Each visual marker 516 is positioned within the image 500 at a respective position which allows the user to identify the angle associated with the respective angle box 506.

In one embodiment, the images 500 and 400 are displayed on different displays. In another embodiment, the images 500 and 400 are displayed on the same display side-by-side for example.

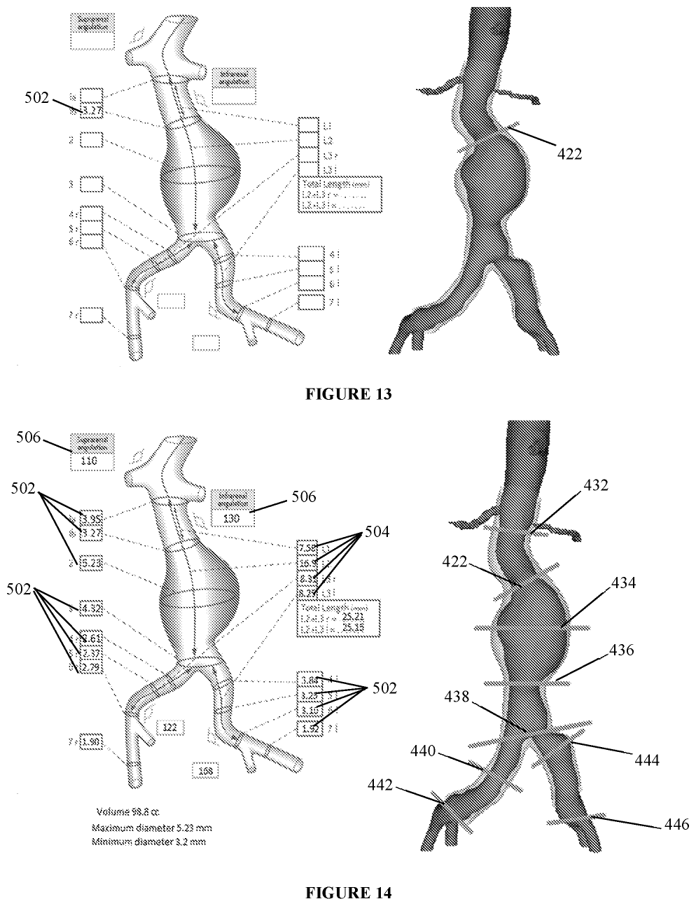

In operation and as illustrated in FIG. 13, the user starts by selecting in the image 500 a given diameter box 502 and the identification of the selected diameter box 502 is sent to the server. The user then selects in the image 400 a desired position for the reference point associated with the selected diameter box. The position of the point selected in the image 400 is then sent to the server. Upon reception of the position of the selected point, the server generates the reference marker 422 and inserts it into the image 400, as described above. Furthermore, the server calculates the diameter of the anatomy at the received position, as described above. The calculated diameter is then displayed in the diameter box 502 previously selected by the user in the image 500.

The operation is repeated for each diameter box 502. The distance boxes 504 and the angle boxes 506 are populated automatically as the user selects the different diameters 502 and the position for their associated reference points in the image 400. As soon as the position for two reference points associated with a given distance box 504 has been selected by the user, the distance between these two points is calculated and displayed in the given distance box 504. Similarly, as soon as the position for two reference points associated with a given angle box 506 has been selected by the user and received by the server, the angle associated with these two points is automatically calculated and displayed in the given distance box 506.

In one embodiment, the user may display the rotated image 400 at the calculated neck angle and then rotate the image 400 to determine a more preferred angle, or drag the line projecting up the neck to alter the angle.

FIG. 14 illustrates the images 400 and 500 once the user has selected the position for the reference points associated with all of the diameter boxes 502. In this case, each box 502, 504, 506 includes the value of its associated parameter.

Once the value for all parameters has been determined, the method 300 further comprises a processing step of determining the characteristics for the stent using the determined characteristics of the anatomy.

In one embodiment, the method 300 further comprises the steps of calculating the volume of the aneurysm and/or the minimal and/or maximal diameters of the aneurysm and displaying the calculated volume of the aneurysm and/or minimal and/or maximal diameters of the aneurysm.

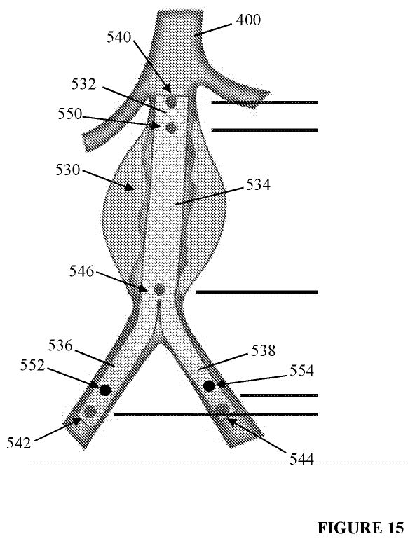

As illustrated in FIG. 15, a stent 530 comprises a neck 532, a body 534, a left leg 536 and a right leg 538, and extends between a top end 540 and a left bottom end 542 at the end of the left leg 536 and a right bottom end 544 at the end of the right leg 538. The left and right legs 536 and 538 of the stent 530 separate at a bifurcation point 546. The neck of the stent 530 extends between the top end 540 and the point 550. The body of the stent extends between the point 550 and the bifurcation point 546. The left leg 536 of the stent 530 extends between the bifurcation point 546 and the left bottom end 542 while the landing area of the left leg 536 extends between the point 552 and the bottom end 542. The right leg 538 of the stent 530 extends between the bifurcation point 546 and the right bottom end 544 while the landing area of the right leg 538 extends between the point 554 and the bottom end 544.

The characteristics of the stent 530 are determined using the determined diameters, lengths and angles described above, as described below. The stent 530 is to be inserted into the subject so that the top end 540 of the stent be aligned with the inflection point of the suprarenal segment identified by visual marker 432, the left bottom end 542 of the stent 530 be aligned with the reference point identified by visual marker 442 and the right bottom end 544 of the stent 530 be aligned with the reference point identified by the visual marker 446.

The suprarenal neck angle and the infrarenal neck angle for the neck of the stent 530 are chosen to be equal to the suprarenal neck angle and the infrarenal neck angle, respectively, determined for the neck of the aneurysm.

The diameter of the stent 530 at the top end 540 is chosen based on the diameter of the aorta determined at the inflection point of the suprarenal segment identified by the visual marker 432 and the diameter of the bottom of the neck 550 of the stent 530, i.e. the diameter at point 550, is chosen based on the diameter determined for the bottom of a neck of the aneurysm identified by the visual marker 422. In one embodiment, the diameter of the stent 530 at the top end 540 is chosen to be equal to the diameter of the aorta determined at the inflection point of the suprarenal segment.

In another embodiment, the diameter of the stent 530 at the top end 540 is chosen to be slightly larger than the diameter of the aorta determined at the inflection point of the suprarenal segment. For example, the diameter of the stent 530 at the top end 540 is chosen to be about 20% larger than the diameter of the aorta determined at the inflection point of the suprarenal segment. Similarly, in one embodiment, the diameter of the bottom of the neck 550 of the stent 530 is chosen to be equal to the diameter determined for the bottom of a neck of the aneurysm. In another embodiment, the diameter of the bottom of the neck 550 of the stent 530 is chosen to be slightly larger than the diameter determined for the bottom of a neck of the aneurysm. For example, the diameter of the bottom of the neck 550 of the stent 530 is chosen to be about 20% larger than the diameter determined for the bottom of a neck of the aneurysm.

In one embodiment, the diameter of the neck 532 varies along the length thereof so that the neck 532 is provided with a flared shape.

The length of the neck 532 of the stent 530 is chosen to be equal to the determined distance between the inflection point of the suprarenal segment identified by the visual marker 432 and the bottom of a neck of the aneurysm identified by reference point 422.

The diameter of the left leg 536 at the left bottom end 542 is chosen based on the diameter of the left common iliac artery determined at reference point 442 and the diameter of the left leg 536 at the point 552 is chosen based on the diameter of the left common iliac determined at reference point 440. In one embodiment, the diameter of the left leg 536 at the left bottom end 542 is chosen to be equal to the diameter of the left common iliac artery determined at reference point 442. In another embodiment, the diameter of the left leg 536 at the left bottom end 542 is chosen to be slightly larger than the diameter of the left common iliac artery determined at reference point 442. For example, the diameter of the left leg 536 at the left bottom end 542 is chosen to be about 20% larger than the diameter of the left common iliac artery determined at reference point 442. Similarly, the diameter of the left leg 536 at the point 552 may be chosen to be equal to the diameter of the left common iliac determined at reference point 440. In another embodiment, the diameter of the left leg 536 at the point 552 is chosen to be slightly larger than the diameter of the left common iliac determined at reference point 440. For example, the diameter of the left leg 536 at the point 552 may be chosen to be about 20% larger than the diameter of the left common iliac determined at reference point 440.

In one embodiment, the diameter of the left leg 536 varies along the length thereof so that the left leg 536 is provided with a flared shape.

The length of the landing area of the left leg 536 of the stent 530 is chosen to be equal to the determined distance between the reference points identified by visual marker 440 and 442.

The diameter of the right leg 538 at the right bottom end 544 is chosen based on the diameter of the right common iliac artery determined at reference point 446 and the diameter of the right leg 538 at the point 554 is chosen based on the diameter of the right common iliac determined at reference point 444. In one embodiment, the diameter of the right leg 538 at the right bottom end 544 is chosen to be equal to the diameter of the right common iliac artery determined at reference point 446. In another embodiment, the diameter of the right leg 538 at the right bottom end 544 is chosen to be slightly larger than the diameter of the right common iliac artery determined at reference point 446. For example, the diameter of the right leg 538 at the right bottom end 544 is chosen to be about 20% larger than the diameter of the right common iliac artery determined at reference point 446. In one embodiment, the diameter of the right leg 538 at the point 554 is chosen to be equal to the diameter of the right common iliac determined at reference point 444. In another embodiment, the diameter of the right leg 538 at the point 554 is chosen to be slightly larger than the diameter of the right common iliac determined at reference point 444. For example, the diameter of the right leg 538 at the point 554 is chosen to be about 20% larger than the diameter of the right common iliac determined at reference point 444.

In one embodiment, the diameter of the right leg 538 varies along the length thereof so that the right leg 538 is provided with a flared shape.

The length of the landing area of the right leg 538 of the stent 530 is chosen to be equal to the determined distance between the reference points identified by visual marker 444 and 446.

In one embodiment, the diameter of the body 534 of the stent 530 is chosen to be equal to the determined diameter for the bottom of the neck identified by the visual marker 422.

In one embodiment, the length of the body 534 of the stent 530, i.e. the distance between the bottom end 550 of the neck 532 and the bifurcation point 546, is predefined or selected according to characteristics of the subject such as the height of the subject. In this case, the length of the left leg 536 is determined using the length of the body 534 and the determined fourth length described above. The length of the right leg 538 of the stent 530 is determined using the length of the body 534 and the determined fifth length described above.

In another embodiment, the length of the body 534 of the stent 530 is chosen to be at most equal to a predefined maximal length. For example, the length of the body 534 may be chosen to be comprised between the distance between the bottom of the neck identified by the visual marker 422 and the bottom of the aneurysm identified by the visual marker 436 and the distance between the bottom of the neck identified by the visual marker 422 and the bifurcation point identified by the visual marker 438 while being less than the predefined maximal length.

In one embodiment, once the characteristics for the stent has been determined, the method 430 further comprises a processing step of generating a 3D model of a stent 530 based on the determined characteristics, generating an image of a stent 530 based on the 3D model of the stent and combining the image of the stent with the image 400 of the anatomy to allow the user see the stent 530 in situ. For example, the image of the stent 530 may be superimposed on the image 400 as illustrated in FIG. 15. In this case, the image of the stent 530 is superimposed on the image 400 so that the top end 532 of the stent 530 be aligned with the inflection point of the suprarenal segment identified by the visual marker 432 in the image 400, the left bottom end 542 of the stent 530 be aligned with the reference point on the left common iliac artery identified by the visual marker 442 and the right bottom end 544 of the stent 530 be aligned with the reference point on the right common iliac artery identified by the visual marker 446.

In one embodiment, the image of the stent and the image 400 are 3D images so that the combined image is also a 3D image. In one embodiment, the user is allowed to manipulate the 3D combined image, e.g., the user may rotate, enlarge and/or the like the 3D combined image. The user may also move one image relative to the other, e.g., the user may move the image of the stent relative to the image 400 within the combined image.

In one embodiment, predefined different stents and/or predefined stent pieces are stored in a database each along with a respective image. A stent piece may correspond to a neck of a stent, a body of a stent, a leg, an assembly comprising a body and a leg of a stent, and/or the like. When the database comprises stent pieces, a stent is designed by selecting different stent pieces that when assembled together, form a stent of which the characteristics substantially correspond to the determined characteristics. For example, the database may comprise a first set of predefined stent necks, a second set of predefined stent bodies, a third set of left legs and a fourth set of right legs. In this case, a stent is designed by selecting a given neck from the first set, a given body from the second set, a left leg from the third set and a right leg from the fourth set, based on the determined characteristics for the stent, so that the selected neck, body, left leg and right leg form together a selected stent of which the characteristics substantially correspond to the determined characteristics for the stent.

In an embodiment in which the database comprises predefined stents, each predefined stents is provided with a respective geometry and a respective configuration. The method 300 comprises the processing step of selecting amongst the predefined stent, a given stent based on the determined characteristics for the stent, e.g., selecting the predefined stent of which the characteristics correspond the most to the determined characteristics for the stent. The given selected predefined stent corresponds to the predefined stent of which the characteristics best match the determined characteristics for the stent 530. The image corresponding to the selected predefined stent is retrieved from the database and combined with the image 400 of the anatomy before being provided for display to the user.

In an embodiment in which the database comprises predefined stent pieces, the method 300 comprises the processing step of selecting amongst the predefined pieces, the given pieces that best match the characteristics determined for the stent 530. For example, a given neck is selected amongst predefined necks based on the characteristics determined for the neck 532 of the stent 530, i.e., the given predefined neck of which the characteristics best match the determined characteristics for the neck 532 is chosen. A body, a left leg and a right leg are also selected based on the determined characteristics for the stent 530. The images associated with the selected stent bodies are retrieved from the database and combined together to form the image of a stent which is combined with the image 400 before being displayed to the user.

In one embodiment, the selection of the adequate stent pieces amongst the database is performed using an adequate machine learning algorithm trained for this purpose, or any other adequate artificial intelligence technique.

It should be understood that the determined characteristics for the stent 530 are outputted. For example, they may be stored in memory.

In one embodiment, the image of the stent displayed to the user is interactive, i.e. the user may modify the image in order to modify the characteristics of the stent 530. Upon modification of the image of the stent 530 by the user, the characteristics of the stent 530 are automatically updated.

It should be understood that any adequate 3D model of the anatomy of a subject comprising at least a portion of a suprarenal segment of a thoracic aorta, an aneurysm on an infrarenal segment of the thoracic aorta, an aortic bifurcation and at least a portion of a left common iliac artery and a right common iliac artery may be used. For example, the 3D model of the anatomy may be a 3D mesh model, a RAW map, a strain map, an anatomy map, an ILT map, a growth map or the like obtained from medical images taken by a medical imaging apparatus.

In one embodiment, the user may switch from one model to another and the markers present in the initial model are reproduced on the second model at the exact same position. For example, the displayed image 400 may be the 3D mesh model of an aorta. The user then selects to display the RAW map for the same aorta. The markers present on the 3D mesh model and selected by the user are then reproduced on the RAW map at the exact same location.

### Medical Imaging Apparatus

The medical imaging apparatus is configured to inter alia (i) acquire, according to acquisition parameters, one or more images comprising the anatomy of a given subject during a cardiac cycle; and (ii) transmit the images to a database.

The medical imaging apparatus may comprise one of: a computed tomography (CT) scanner, a magnetic resonance imaging (MRI) scanner, a ultrasound and the like.

In some embodiments of the present technology, the medical imaging apparatus may comprise a plurality of medical imaging apparatuses, such as one or more of a computational tomography (CT) scanner, a magnetic resonance imaging (MRI) scanner, a ultrasound, and the like.

The medical imaging apparatus may be configured with specific acquisition parameters for acquiring images of the patient comprising the aorta of the patient.

As a non-limiting example, in one or more embodiments where the medical imaging apparatus is implemented as a CT scanner, a CT protocol comprising pre-operative retrospectively gated multidetector CT (MDCT-64-row multi-slice CT scanner) with variable dose radiation to capture the R-R interval may be used.

As another non-limiting example, in one or more embodiments where the medical imaging procedure comprises a MRI scanner, the MR protocol can comprise steady state T2 weighted fast field echo (TE=2.6 ms, TR=5.2 ms, flip angle 110 degree, fat suppression (SPIR), echo time 50 ms, maximum 25 heart phases, matrix 256×256, acquisition voxel MPS (measurement, phase and slice encoding directions) 1.56/1.56/3.00 mm and reconstruction voxel MPS 0.78/0.78/1.5), or similar cine acquisition of the portion of aorta under study, axial slices.

In one or more embodiments, the medical imaging apparatus is part of a Picture Archiving and Communication System (PACS) for storing and retrieving medical images together with the workstation computer and other electronic devices such as the server 230.

### Mesh Acquisition Procedure

The mesh acquisition procedure is configured to acquire a 3D mesh of the aorta of the patient having been generated based on a multislice image stack acquired by a medical imaging apparatus.

In one or more embodiments, the mesh acquisition procedure generates the 3D mesh of the anatomy of the patient.

In one or more other embodiments, the 3D mesh of the anatomy may have been generated by another electronic device, and the mesh acquisition procedure may obtain the 3D mesh from the electronic device (not depicted).

In one or more embodiments, the mesh acquisition procedure uses a 3D mesh having been generated as an intermediate step during a generation procedure. For example, the generation procedure may have been used to generate, based on the multiphase image stack, one or more of: a strain map (including the strain heterogeneity map and the strain/relative deformation map), a regional aortic weakness (RAW) map, an intraluminal thrombus (ILT) map, and a calcification distribution map.

### Map Generation Procedure

The generation procedure receives the multiphase image stack of a given patient. The multiphase image stack comprises images of an anatomy of a patient having been acquired during a cardiac cycle, the patient suffering from an aortic aneurysm.

In one or more embodiments, the multiphase image stack is received from the medical imaging apparatus 210 and/or the workstation computer 215.

### Strain Map

The generation procedure generates, based on the multiphase stack 410, a strain map.

In one or more embodiments, to generate the pre-surgical strain map based on the medical images, the generation procedure uses the methods and systems disclosed in PCT Patent Application No. PCT/IB2020/059018 filed on Sep. 25, 2020 by the same applicant, the entirety of which is incorporated herein by reference.

### Strain Relative Deformation Map

In one or more embodiments, the generation procedure generates, based on the strain map, a strain and relative deformation map.

The strain and relative deformation map comprise measures of local deformation of the aortic wall during the cardiac cycle.

The strain relative deformation map is used evaluate the strain in the aorta relative to “healthier” regions, to show areas of relative strength or weakness in order to support decision for optimal stent placement.

### Strain Heterogeneity Map

In one or more embodiments, the generation procedure generates, based on the strain map, a strain heterogeneity map.

The strain heterogeneity map is indicative of a level of heterogeneity in strain distribution in the aorta, and shows regions where strain varies compared to other regions, and where the regions may be determined based on different strain ranges.

The strain heterogeneity map is used to evaluate the level of heterogeneity of the strain distribution (i.e., very heterogeneous regions may affect the stent sealing and placement).

### Regional Aortic Weakness (RAW) Map

The generation procedure generates, based on the multiphase image stack, a RAW map.

In one or more embodiments, to generate the pre-surgical RAW map based on the medical images, the generation procedure uses the methods and systems disclosed in PCT Patent Application No. PCT/IB2020/059018 filed on Sep. 25, 2020 by the same applicant, the entirety of which is incorporated herein by reference.

In one or more alternative embodiments, the pre-surgical RAW map may be optional.

### Calcification Distribution Map and Intraluminal Thrombus Thickness Map

The generation procedure accesses the set segmentation ML models. The segmentation ML models are used to detect calcium deposits within the aorta, iliac arteries, the lumen, and remaining tissues including wall and intraluminal thrombus (ILT) in the medical images of each patient. The choice of reference points may be optimized to avoid calcium deposits.

In one or more embodiments, to detect the aorta, iliac arteries, the lumen, and remaining tissues including wall and intraluminal thrombus (ILT) in medical images, the generation procedure uses the methods and systems disclosed in U.S. Provisional Patent Application Ser. No. 63/152,105 filed on Feb. 22, 2021, by the same applicant, the entirety of which is incorporated herein by reference.

The generation procedure generates, using the set of segmentation ML models 280, based on the multiphase stack, an intraluminal thrombus thickness (ILT) map and calcification distribution map.

In one or more alternative embodiments, the aortic wall weakness values at the sealing zones may be obtained without using the pre-surgical RAW map. As a non-limiting example, in embodiments where the pre-surgical RAW map is not used, the aortic wall weakness values at the sealing zones may be received from another electronic device.

In one or more other embodiments, the 3D mesh and one or more of the regional aortic weakness (RAW) map, the strain heterogeneity map, the strain/relative deformation map, the intraluminal thrombus thickness (ILT) map and the calcification distribution map may be generated by one or more other electronic devices executing the generation procedure and then received by the mesh acquisition procedure.

In one or more alternative embodiments, the mesh acquisition procedure outputs the 3D mesh and one or more of the regional aortic weakness (RAW) map, the strain heterogeneity map, the strain/relative deformation map, the intraluminal thrombus thickness (ILT) map and the calcification distribution map.

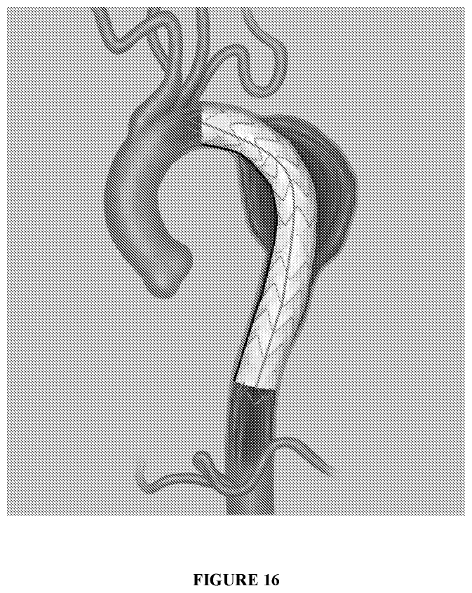

It should be understood that the above-described method and system may be used for designing other type of stents. For example, they may be used for designing the stent for a thoracic aortic aneurysm illustrated in FIG. 16. In this case, the method comprises the steps of:
providing for display a first image of a 3D model of at least a portion of the thoracic aorta;
receiving a position for each one of a plurality of reference points chosen along the thoracic aorta;
inserting in the first image a visual marker at the position of each reference point, thereby obtaining a second image;
providing the second image for display;
for each reference point, calculating a respective diameter;
calculating at least one length using the positions of the reference points;
optionally calculating a curvature of thoracic aorta; and
outputting the calculated diameters, the calculated length(s) and optionally the curvature.

The method further comprises generating an image of the stent using the calculated parameters and combining the image of the stent with the image of the thoracic aorta before display.

It should be expressly understood that not all technical effects mentioned herein need to be enjoyed in each and every embodiment of the present technology. For example, embodiments of the present technology may be implemented without the user enjoying some of these technical effects, while other non-limiting embodiments may be implemented with the user enjoying other technical effects or none at all.

Modifications and improvements to the above-described implementations of the present technology may become apparent to those skilled in the art. The foregoing description is intended to be exemplary rather than limiting.

What is claimed is:

1. A computer-implemented method for designing a stent, the computer-implemented method comprising:
providing for display a first image of a 3D model of a body part of a subject, the body part comprising at least an infrarenal segment of a thoracic aorta, an aneurysm on the infrarenal segment of the thoracic aorta, an aortic bifurcation and at least a portion of a left common iliac artery and a right common iliac artery;
receiving a position for each one of a plurality of reference points, the reference points comprising at least an inflection point of the suprarenal segment, a bottom of a neck of the aneurysm, a bottom of the aneurysm, a first point and a second point on the left common iliac artery and a first and a second point on the right common iliac artery;
inserting in the first image a visual marker at the position for each reference point, thereby obtaining a second image;
providing the second image for display;
for each reference point, calculating a respective diameter;
calculating a first length of the neck using the position of the inflection point of the suprarenal segment and the position of the bottom of a neck of the aneurysm, a second length of a left landing area using the position of the first point and second points on the left common iliac artery, a third length of a right landing area using the position of the first point and second points on the right common iliac artery, a fourth length of a left leg of the stent and a fifth length of a right leg of the stent using the position of some of the reference points;
calculating a suprarenal neck angle using the position of the inflection point of the suprarenal segment and the position of the bottom of the neck of the aneurysm, and an infrarenal neck angle using the position of the bottom of the neck of the aneurysm and the position of the bottom of the aneurysm; and
outputting the respective diameter for each reference point, the first, second, third, fourth and fifth lengths, the suprarenal neck angle and the infrarenal neck angle.

2. (canceled)

3. The computer-implemented method of claim 1, wherein the fourth length is determined using the position of one of the inflection point and the bottom of the neck and the position of one of the first point and the second point on the left common iliac artery, and the fifth length is determined using the position of one of the inflection point and the bottom of the neck and the position of one of the first point and the second point on the right common iliac artery.

4. The computer-implemented method of claim 3, further comprising:
generating an image of a stent using the respective diameter for each reference point, the first, second, third, fourth and fifth lengths, the suprarenal neck angle, the infrarenal neck angle and a given predefined length for a body of the stent;
combining the image of the stent on the image with the first image, thereby obtaining a further image; and
providing the further image for display.

5. The computer-implemented method of claim 1, further comprising receiving a position of a bifurcation point between the left and right common iliac arteries, and calculating and outputting a diameter of the infrarenal segment at the bifurcation point.

6. (canceled)

7. The computer-implemented method of claim 5, further comprising determining a sixth length using the position of one of the inflection point and the bottom of the neck and the position of the bifurcation point, said calculating the fourth length being performed using the position of the bifurcation point and the position of one of the first point and the second point on the left common iliac artery, and said calculating the fifth length being performed using the position of the bifurcation point and the position of one of the first point and the second point on the right common iliac artery.

8. The computer-implemented method of claim 7, further comprising:
generating an image of a stent using the respective diameter for each reference point, the first, second, third, fourth, fifth and sixth lengths, the suprarenal neck angle and the infrarenal neck angle;
combining the image of the stent on the image with the first image, thereby obtaining a further image; and
providing the further image for display.

9. (canceled)

10. The computer-implemented method of claim 1, further comprising:
receiving a new position for the visual marker;
moving the visual marker at the new position within the second image, thereby obtaining an updated image;
providing the updated image for display; and
calculating a further diameter at the new position; and
outputting the further diameter.

11.-12. (canceled)

13. The computer-implemented method of claim 1, further comprising:
generating a reference image of the body part, the reference image comprising at least a portion of a stylized infrarenal segment of the thoracic aorta, a stylized aneurysm, a stylized aortic bifurcation and at least a portion of a first stylized common iliac artery and a second stylized common iliac artery;
inserting a visual indicator for each one of the plurality of the reference points within the reference image; and
providing the reference image for display.

14.-17. (canceled)

18. The computer-implemented method of claim 1, further comprising for each one of the first and second common iliac arteries:
receiving a respective iliac point; and
calculating a respective curvature angle at the respective iliac point; and
providing the respective curvature angle for display.

19.-22. (canceled)

23. The computer-implemented method of claim 4, wherein said generating the image of the stent comprises:
accessing a database comprising predefined stent pieces;
selecting adequate stent pieces amongst the predefined stent pieces using the respective diameter for each reference point, the first, second, third, fourth and fifth lengths, the suprarenal neck angle, the infrarenal neck angle and the given predefined length for the body of the stent, the adequate stent pieces forming together the stent; and
generating the image of the stent using images of the adequate stent pieces.

24.-25. (canceled)

26. A system for designing a stent, the system comprising:
a processor; and
a non-transitory computer readable storage medium comprising instructions stored thereon;
the processor, upon execution of the instructions, being configured for:
providing for display a first image of a 3D model of a body part of a subject, the body part comprising at least an infrarenal segment of a thoracic aorta, an aneurysm on the infrarenal segment of the thoracic aorta, an aortic bifurcation and at least a portion of a left common iliac artery and a right common iliac artery;
receiving a position for each one of a plurality of reference points, the reference points comprising at least an inflection point of the suprarenal segment, a bottom of a neck of the aneurysm, a bottom of the aneurysm, a first point and a second point on the left common iliac artery and a first and a second point on the right common iliac artery;
inserting in the first image a visual marker at the position for each reference point, thereby obtaining a second image;
providing the second image for display;
for each reference point, calculating a respective diameter;
calculating a first length of the neck using the position of the inflection point of the suprarenal segment and the position of the bottom of a neck of the aneurysm, a second length of a left landing area using the position of the first point and second points on the left common iliac artery, a third length of a right landing area using the position of the first point and second points on the right common iliac artery, a fourth length of a left leg of the stent and a fifth length of a right leg of the stent using the position of some of the reference points;
calculating a suprarenal neck angle using the position of the inflection point of the suprarenal segment and the position of the bottom of the neck of the aneurysm, and an infrarenal neck angle using the position of the bottom of the neck of the aneurysm and the position of the bottom of the aneurysm; and
outputting the respective diameter for each reference point, the first, second, third, fourth and fifth lengths, the suprarenal neck angle and the infrarenal neck angle.

27. (canceled)

28. The system of claim 26, wherein the fourth length is determined using the position of one of the inflection point and the bottom of the neck and the position of one of the first point and the second point on the left common iliac artery, and the fifth length is determined using the position of one of the inflection point and the bottom of the neck and the position of one of the first point and the second point on the right common iliac artery.

29. The system of claim 28, further comprising:
generating an image of a stent using the respective diameter for each reference point, the first, second, third, fourth and fifth lengths, the suprarenal neck angle, the infrarenal neck angle and a given predefined length for a body of the stent;
combining the image of the stent on the image with the first image, thereby obtaining a further image; and
providing the further image for display.

30. The system of claim 26, further comprising receiving a position of a bifurcation point between the left and right common iliac arteries and calculating and outputting a diameter of the infrarenal segment at the bifurcation point.

31. (canceled)

32. The system of claim 30, further comprising determining a sixth length using the position of one of the inflection point and the bottom of the neck and the position of the bifurcation point, said calculating the fourth length being performed using the position of the bifurcation point and the position of one of the first point and the second point on the left common iliac artery, and said calculating the fifth length being performed using the position of the bifurcation point and the position of one of the first point and the second point on the right common iliac artery.

33. The system of claim 32, further comprising:
generating an image of a stent using the respective diameter for each reference point, the first, second, third, fourth, fifth and sixth lengths, the suprarenal neck angle and the infrarenal neck angle;
combining the image of the stent on the image with the first image, thereby obtaining a further image; and
providing the further image for display.

34. (canceled)

35. The system of claim 26, further comprising:
receiving a new position for the visual marker;
moving the visual marker at the new position within the second image, thereby obtaining an updated image;
providing the updated image for display;
calculating a further diameter at the new position providing the further diameter for display.

36.-37. (canceled)

38. The system of claim 26, further comprising:
generating a reference image of the body part, the reference image comprising at least a portion of a stylized infrarenal segment of the thoracic aorta, a stylized aneurysm, a stylized aortic bifurcation and at least a portion of a first stylized common iliac artery and a second stylized common iliac artery;
inserting a visual indicator for each one of the plurality of the reference points within the reference image; and
providing the reference image for display.

39.-42. (canceled)

43. The system of claim 26, further comprising for each one of the first and second common iliac arteries:
receiving a respective iliac point; and
calculating a respective curvature angle at the respective iliac point; and
providing the respective curvature angle for display.

44.-47. (canceled)

48. The system of claim 29, wherein said generating the image of the stent comprises:
accessing a database comprising predefined stent pieces;
selecting adequate stent pieces amongst the predefined stent pieces using the respective diameter for each reference point, the first, second, third, fourth and fifth lengths, the suprarenal neck angle, the infrarenal neck angle and the given predefined length for the body of the stent, the adequate stent pieces forming together the stent; and
generating the image of the stent using images of the adequate stent pieces.

49. (canceled)

50. A system for designing a stent, the system comprising:
a processor; and
a non-transitory computer readable storage medium comprising instructions stored thereon;
the processor, upon execution of the instructions, being configured for:
providing for display a first image of a 3D model of at least a portion of a body part comprising an aneurysm;
receiving a position for each one of a plurality of reference points chosen along the body part;
inserting in the first image a visual marker at the position of each reference point, thereby obtaining a second image;
providing the second image for display;
for each reference point, calculating a respective diameter;
calculating at least one length using the positions of the reference points; and
outputting the respective diameter and at least one length.
